(19) United States
(12) Patent Application Publication
(10) Pub. No.: US 2017/0283618 A1
(43) Pub. Date: Oct. 5, 2017

(54) NON-LINEAR DIELECTRIC MATERIALS AND CAPACITOR

(71) Applicant: Capacitor Sciences Incorporated, Menlo Park, CA (US)

(72) Inventors: Pavel Ivan Lazarev, Menlo Park, CA (US); Paul T. Furuta, Sunnyvale, CA (US); Barry K. Sharp, Redwood City, CA (US); Yan Li, Fremont, CA (US)

(21) Appl. No.: 15/469,126

(22) Filed: Mar. 24, 2017

Related U.S. Application Data
(60) Provisional application No. 62/318,134, filed on Apr. 4, 2016.

(51) Int. Cl. C09B 57/00; C09B 7/02; H01G 4/14

(52) U.S. Cl. CPC C09B 57/00; H01G 4/14; C09B 7/02; C07C 69/157

(57) ABSTRACT

A composite organic compound characterized by polarizability and resistivity that has a general structural formula: [symbol]
where C is a chromophore fragment, P is an optionally connected rylene fragment, D and A are electron donating and accepting groups respectively, and R represents resistive substituents optionally connected directly or via dopant connecting groups.

This application claims the benefit of U.S. Provisional Application No. 62/318,134 filed Apr. 4, 2016, which is hereby incorporated herein by reference in its entirety.

### FIELD OF THE INVENTION

The present disclosure relates generally to passive components of electrical circuit and more particularly to a composite organic compound and capacitor based on this material and intended for energy storage.

### BACKGROUND

A capacitor is a passive electronic component that is used to store energy in the form of an electrostatic field, and comprises a pair of electrodes separated by a dielectric layer. When a potential difference exists between the two electrodes, an electric field is present in the dielectric layer. An ideal capacitor is characterized by a single constant value of capacitance, which is a ratio of the electric charge on each electrode to the potential difference between them. For high voltage applications, much larger capacitors have to be used.

A capacitor is an energy storage device that stores an applied electrical charge for a period of time and then discharges it. It is charged by applying a voltage across two electrodes and discharged by shorting the two electrodes. A voltage is maintained until discharge even when the charging source is removed. A capacitor blocks the flow of direct current and permits the flow of alternating current. The energy density of a capacitor is usually less than for a battery, but the power output of a capacitor is usually higher than for a battery. Capacitors are often used for various purposes including timing, power supply smoothing, coupling, filtering, tuning and energy storage. Batteries and capacitors are often used in tandem such as in a camera with a flash. The battery charges the capacitor that then provides the high power needed for a flash. The same idea works in electric and hybrid vehicles where batteries provide energy and capacitors provide power for starting and acceleration.

One important characteristic of a dielectric material is its breakdown field. This corresponds to the value of electric field strength at which the material suffers a catastrophic failure and conducts electricity between the electrodes. For most capacitor geometries, the electric field in the dielectric can be approximated by the voltage between the two electrodes divided by the spacing between the electrodes, which is usually the thickness of the dielectric layer. Since the thickness is usually constant it is more common to refer to a breakdown voltage, rather than a breakdown field. There are a number of factors that can dramatically reduce the breakdown voltage. In particular, the geometry of the conductive electrodes is important factor affecting breakdown voltage for capacitor applications. In particular, sharp edges or points hugely increase the electric field strength locally and can lead to a local breakdown. Once a local breakdown starts at any point, the breakdown will quickly “trace” through the dielectric layer until it reaches the opposite electrode and causes a short circuit.

Breakdown of the dielectric layer usually occurs as follows. Intensity of an electric field becomes high enough to “pull” electrons from atoms of the dielectric material and makes them conduct an electric current from one electrode to another. Presence of impurities in the dielectric or imperfections of the crystal structure can result in an avalanche breakdown as observed in semiconductor devices.

Another important characteristic of a dielectric material is its dielectric permittivity. Different types of dielectric materials are used for capacitors and include ceramics, polymer film, paper, and electrolytic capacitors of different kinds. The most widely used polymer film materials are polypropylene and polyester. Increasing dielectric permittivity allows for increasing volumetric energy density, which makes it an important technical task.

The article “Synthesis and spectroscopic characterization of an alkoxysilane dye containing C. I. Disperse Red 1” (Yuanjing Cui, Minquan Wang, Lujian Chen, Guodong Qian, Dyes and Pigments, 62 (2004) pp. 43-47) describe the synthesis of an alkoxysilane dye (ICTES-DR1) which was copolymerized by sol-gel processing to yield organic-inorganic hybrid materials for use as second-order nonlinear optical (NLO) effect. C. I. Disperse Red 1 (DR1) was attached to Si atoms by a carbamate linkage to provide the functionalized silane via the nucleophilic addition reaction of 3-isocyanatopropyl triethoxysilane (ICTES) with DR1 using triethylamine as catalyst. The authors found that triethylamine and dibutyltin dilaurate were almost equally effective as catalysts. The physical properties and structure of ICTES-DR1 were characterized using elemental analysis, mass spectra, 1 H-NMR, FTIR, UV-visible spectra and differential scanning calorimetry (DSC). ICTES-DR1 displays excellent solubility in common organic solvents.

Second-order nonlinear optical (NLO) effects of organic molecules have been extensively investigated for their advantages over inorganic crystals. Properties studied, for example, include their large optical non-linearity, ultra fast response speed, high damage thresholds and low absorption loss, etc. Particularly, organic thin films with excellent optical properties have tremendous potential in integrated optics such as optical switching, data manipulation and information processing. Among organic NLO molecules, azo-dye chromophores have been a special interest to many investigators because of their relatively large molecular hyper-polarizability (b) due to delocalization of the p-electronic clouds. They were most frequently either incorporated as a guest in the polymeric matrix (guest-host polymers) or grafted into the polymeric matrix (functionalized polymers) over the past decade.

Chromophoric orientation is obtained by applying a static electric field or by optical poling. Whatever the poling process, poled-order decay is an irreversible process which tends to annihilate the NLO response of the materials and this process is accelerated at higher temperature. For device applications, the most probable candidate must exhibit inherent properties that include: (i) high thermal stability to withstand heating during poling; (ii) high glass transition temperature (T_{g}) to lock the chromophores in their acentric order after poling.

Most of the polymers, however, have either low T_{g} or poor thermal stability which makes them unsuitable for direct use. To overcome these problems, one attractive approach is incorporating the nonlinear optical active chromophore into a polymerizable silane by covalent bond to yield an alkoxysilane dye which can be copolymerized via sol-gel processing to form organic-inorganic hybrid materials. The hydrolysis and condensation of functionalized silicon alkoxydes can yield a rigid amorphous three-dimensional network which leads to slower relaxation of NLO chromophores. Therefore, sol-gel hybrid nonlinear optical materials have received significant attention and exhibited the desired properties. In this strategy, the design and synthesis of new network-forming alkoxysilane dye are of paramount importance and detailed investigation of them will offer great promise in the fabrication of new materials for second-order nonlinear optics that will eventually meet the basic requirements in building photonic devices.

In the article “Design and Characterization of Molecular Nonlinear Optical Switches” (Frederic Castet et. al., ACCOUNTS OF CHEMICAL RESEARCH, pp. 2656-2665, (2013), Vol. 46, No. 11), Castet et. al. illustrate the similarities of the experimental and theoretical tools to design and characterize highly efficient NLO switches but also the difficulties in comparing them. After providing a critical overview of the different theoretical approaches used for evaluating the first hyperpolarizabilities, Castet et. al. reported two case studies in which theoretical simulations have provided guidelines to design NLO switches with improved efficiencies. The first example presents the joint theoretical/experimental characterization of a new family of multi-addressable NLO switches based on benzazolo-oxazolidine derivatives. The second focuses on the photoinduced commutation in merocyanine-spiropyran systems, where the significant NLO contrast could be exploited for metal cation identification in a new generation of multiusage sensing devices. Finally, Castet et. al. illustrated the impact of environment on the NLO switching properties, with examples based on the keto-enol equilibrium in aniline derivatives. Through these representative examples, Castet et. al. demonstrated that the rational design of molecular NLO switches, which combines experimental and theoretical approaches, has reached maturity. Future challenges consist in extending the investigated objects to supramolecular architectures involving several NLO-responsive units, in order to exploit their cooperative effects for enhancing the NLO responses and contrasts.

Two copolymers of 3-alkylthiophene (alkyl=hexyl, octyl) and a thiophene functionalized with disperse red 19 (TDR19) as chromophore side chain were synthesized by oxidative polymerization by Marilú Chávez-Castillo et. al. (“Third-Order Nonlinear Optical Behavior of Novel Polythiophene Derivatives Functionalized with Disperse Red 19 Chromophore”, Hindawi Publishing Corporation International Journal of Polymer Science, Volume 2015, Article ID 219361, 10 pages, http://dx.doi.org/10.1155/2015/219361). The synthetic procedure was easy to perform, cost-effective, and highly versatile. The molecular structure, molecular weight distribution, film morphology, and optical and thermal properties of these polythiophene derivatives were determined by NMR, FT-IR, UV-Vis GPC, DSC-TGA, and AFM. The third-order nonlinear optical response of these materials was performed with nanosecond and femtosecond laser pulses by using the third-harmonic generation (THG) and Z-scan techniques at infrared wavelengths of 1300 and 800 nm, respectively. From these experiments it was observed that although the TRD19 incorporation into the side chain of the copolymers was lower than 5%, it was sufficient to increase their nonlinear response in solid state. For instance, the third-order nonlinear electric susceptibility of solid thin films made of these copolymers exhibited an increment of nearly 60% when TDR19 incorporation increased from 3% to 5%. In solution, the copolymers exhibited similar two-photon absorption cross sections σ_{2PA} with a maximum value of 8545 GM and 233 GM (1GM=10⁻⁵⁰ cm⁴s) per repeated monomeric unit.

The theory of molecular nonlinear optics based on the sum-over-states (SOS) model was reviewed by Mark G. Kuzyk et. al. (“Theory of Molecular Nonlinear Optics”, Advances in Optics and Photonics 5, 4-82 (2013) doi: 10.1364/AOP .5.000004). The interaction of radiation with a single wtp-isolated molecule was treated by first-order perturbation theory, and expressions were derived for the linear (αᵢⱼ) polarizability and nonlinear (βᵢⱼₖ, γᵢⱼₖₗ) molecular hyperpolarizabilities in terms of the properties of the molecular states and the electric dipole transition moments for light-induced transitions between them. Scale invariance was used to estimate fundamental limits for these polarizabilities. The crucial role of the spatial symmetry of both the single molecules and their ordering in dense media, and the transition from the single molecule to the dense medium case (susceptibilities χ⁽¹⁾ᵢⱼ, χ(2)ᵢⱼₖ, χ⁽³⁾ᵢⱼₖₗ) is discussed. For example, for βᵢⱼₖ, symmetry determines whether a molecule can support second-order nonlinear processes or not. For non-centrosymmetric molecules, examples of the frequency dispersion based on a two-level model (ground state and one excited state) are the simplest possible for βᵢⱼₖ and examples of the resulting frequency dispersion were given. The third-order susceptibility is too complicated to yield simple results in terms of symmetry properties. It will be shown that whereas a two-level model suffices for non-centrosymmetric molecules, symmetric molecules require a minimum of three levels in order to describe effects such as two-photon absorption. The frequency dispersion of the third-order susceptibility will be shown and the importance of one and two-photon transitions will be discussed.

The promising class of (polypyridine-ruthenium)-nitrosyl complexes capable of high yield Ru—NO/Ru—ON isomerization has been targeted as a potential molecular device for the achievement of complete NLO switches in the solid state by Joelle Akl, Chelmia Billot et. al. (“Molecular materials for switchable nonlinear optics in the solid state, based on ruthenium-nitrosyl complexes”, New J. Chem., 2013, 37, 3518-3527). A computational investigation conducted at the PBE0/6-31+G** DFT level for benchmark systems of general formula [R-terpyridine-Ru^{II}Cl₂(NO)](PF₆) (R being a substituent with various donating or withdrawing capabilities) lead to the suggestion that an isomerization could produce a convincing NLO switch (large value of the β_{ON}/β_{OFF} ratio) for R substituents of weak donating capabilities. Four new molecules were obtained in order to test the synthetic feasibility of this class of materials with R=4′-p-bromophenyl, 4′-p-methoxyphenyl, 4′-p-diethylaminophenyl, and 4′-p-nitrophenyl. The different cis-(Cl,Cl) and trans-(Cl,Cl) isomers can be separated by HPLC, and identified by NMR and X-ray crystallographic studies.

Single crystals of doped aniline oligomers can be produced via a simple solution-based self-assembly method (see Yue Wang et. al., “Morphological and Dimensional Control via Hierarchical Assembly of Doped Oligoaniline Single Crystals”, J. Am. Chem. Soc. 2012, v. 134, pp. 9251-9262). Detailed mechanistic studies reveal that crystals of different morphologies and dimensions can be produced by a “bottom-up” hierarchical assembly where structures such as one-dimensional (1-D) nanofibers can be aggregated into higher order architectures. A large variety of crystalline nanostructures including 1-D nanofibers and nanowires, 2-D nanoribbons and nanosheets, 3-D nanoplates, stacked sheets, nanoflowers, porous networks, hollow spheres, and twisted coils can be obtained by controlling the nucleation of the crystals and the non-covalent interactions between the doped oligomers. These nanoscale crystals exhibit enhanced conductivity compared to their bulk counterparts as well as interesting structure-property relationships such as shape-dependent crystallinity. Further, the morphology and dimension of these structures can be largely rationalized and predicted by monitoring molecule-solvent interactions via absorption studies. Using doped tetraaniline as a model system, the results and strategies presented by Yue Wang et. al. provide insight into the general scheme of shape and size control for organic materials.

Hu Kang et. al. detail the synthesis and chemical/physical characterization of a series of unconventional twisted π-electron system electro-optic (EO) chromophores (“Ultralarge Hyperpolarizability Twisted π-Electron System Electro-Optic Chromophores: Synthesis, Solid-State and Solution-Phase Structural Characteristics, Electronic Structures, Linear and Nonlinear Optical Properties, and Computational Studies”, J. AM. CHEM. SOC. 2007, vol. 129, pp. 3267-3286). Crystallographic analysis of these chromophores reveals large ring-ring dihedral twist angles (80-89°) and a highly charge-separated zwitterionic structure dominating the ground state. NOE NMR measurements of the twist angle in solution confirm that the solid-state twisting persists essentially unchanged in solution. Optical, IR, and NMR spectroscopic studies in both the solution phase and solid state further substantiate that the solid-state structural characteristics persist in solution. The aggregation of these highly polar zwitterions is investigated using several experimental techniques, including concentration-dependent optical and fluorescence spectroscopy and pulsed field gradient spin-echo (PGSE) NMR spectroscopy in combination with solid-state data. These studies reveal clear evidence of the formation of centrosymmetric aggregates in concentrated solutions and in the solid state and provide quantitative information on the extent of aggregation. Solution-phase DC electric-field-induced second-harmonic generation (EFISH) measurements reveal unprecedented hyperpolarizabilities (nonresonant μβ as high as −488,000×10⁻⁴⁸ esu at 1907 nm). Incorporation of these chromophores into guest-host poled polyvinylphenol films provides very large electro-optic coefficients (r₃₃) of ˜330 pm/V at 1310 nm. The aggregation and structure-property effects on the observed linear/nonlinear optical properties were discussed. High-level computations based on state-averaged complete active space self-consistent field (SA-CASSCF) methods provide a new rationale for these exceptional hyperpolarizabilities and demonstrate significant solvation effects on hyperpolarizabilities, in good agreement with experiment. As such, this work suggests new paradigms for molecular hyperpolarizabilities and electro-optics.

U.S. Pat. No. 5,395,556 (Tricyanovinyl Substitution Process for NLO Polymers) demonstrate NLO effect of polymers that specifies a low dielectric constant. U.S. patent application Ser. No. 11/428,395 (High Dielectric, Non-Linear Capacitor) develops high dielectric materials with non-linear effects. It appears to be an advance in the art to achieve non-linear effects through supramolecular chromophore structures that are insulated from each other that include doping properties in the connecting insulating or resistive elements to the composite organic compound. It further appears to be an advance in the art to combine composite organic compounds with non-linear effects that form ordered structures in a film and are insulated from each other and do not rely on forming self-assembled monolayers on a substrate electrode.

Capacitors as energy storage device have well-known advantages versus electrochemical energy storage, e.g. a battery. Compared to batteries, capacitors are able to store energy with very high power density, i.e. charge/recharge rates, have long shelf life with little degradation, and can be charged and discharged (cycled) hundreds of thousands or millions of times. However, capacitors often do not store energy in small volume or weight as in case of a battery, or at low energy storage cost, which makes capacitors impractical for some applications, for example electric vehicles. Accordingly, it may be an advance in energy storage technology to provide capacitors of higher volumetric and mass energy storage density and lower cost.

A need exists to improve the energy density of capacitors while maintaining the existing power output. There exists a further need to provide a capacitor featuring a high dielectric constant sustainable to high frequencies where the capacitance is voltage dependent. Such a capacitor is the subject of the present disclosure. The capacitor of the present disclosure builds on past work on non-linear optical chromophores and non-linear capacitors comprising said chromophores.

In high frequency applications, it is often important that the capacitors used do not have high dielectric losses. In the case of ferroelectric ceramic capacitors with a high dielectric constant, the presence of domain boundaries and electrostriction provide loss mechanisms that are significant. In contrast, the high dielectric mechanism disclosed in this disclosure involves the movement of an electron in a long molecule and its fixed donor. This occurs extremely rapidly so that losses even at gigahertz frequencies are small.

A second very useful property of the type of capacitor disclosed in the disclosure is its non-linearity. In many applications, it is desirable to have a voltage sensitive capacitance to tune circuits and adjust filters. The disclosed capacitors have such a property; as the mobile electron moves to the far end of the chromophore as the voltage increases, its motion is stopped so that with additional voltage little change in position occurs. As a consequence, the increase in the electric moment of the dielectric is reduced resulting in a diminished dielectric constant.

A third useful property of the type of capacitor disclosed in the disclosure is its resistivity due to ordered resistive tails covalently bonded to the composite organic compound. In many instances, electron mobility is hindered by a matrix of resistive materials. Ordered resistive tails can enhance the energy density of capacitors by increasing the density of polarization units in organized structures such as lamella or micellular structures, while also limiting mobility of electrons on the chromophores. The ordered resistive tails can also crosslink to further enhance the structure of the dielectric film which can reduce localized film defects that can enhance the film's breakdown voltage or field. Further, ordered resistive tails can improve solubility of the composite compound in organic solvents. Still further, the ordered resistive tails act to hinder electro-polar interactions between supramolecular structures formed from pi-pi stacking of the optionally attached polycyclic conjugated molecule.

Said polycyclic conjugated molecule may be a perylene or other combination of rylene fragments.

A fourth very useful property of the type of capacitor disclosed in the disclosure is enhancing the non-linear response of the chromophores by using non-ionic dopant groups to change electron density of the chromophores. Manipulation of the electron density of the chromophores can significantly increase the non-linear response which is useful for increasing the polarization of the capacitor and thus energy density of said capacitor. However, placement and type of dopant groups on chromophores is also important to achieving enhanced non-linear polarization versus a neutral or deleterious effect on the non-linearity of the chromophore.

A fifth very useful property of the type of capacitor disclosed in the disclosure is enhancing the non-linear response of the chromophores by using non-ionic dopant connecting groups to change electron density of the chromophores. Manipulation of the electron density of the chromophores can significantly increase the non-linear response which is useful for increasing the polarization of the capacitor and thus energy density of said capacitor. However, placement and type of dopant connecting groups on chromophores is also important to achieving enhanced non-linear polarization versus a neutral or deleterious effect on the non-linearity of the chromophore.

### BRIEF DESCRIPTION OF THE SEVERAL VIEWS OF THE DRAWING

FIG. 1 shows a composite organic compound comprising a chromophore, two dopant groups and two resistive tails.

FIG. 2A shows a capacitor with two electrodes and a dielectric according to an aspect of the present disclosure.

FIG. 2B shows a coiled film capacitor according to an aspect of the present disclosure.

### SUMMARY

According to an aspect of the present disclosure, a composite organic compound characterized by polarizability and resistivity has a general structural formula:
C is a chromophore fragment comprising an aromatic substituent independently selected from the group consisting of six-membered aromatic rings, five-membered heteroaromatic rings, fused ring systems containing at least one six-membered aromatic ring, and fused ring systems containing at least one five-membered heteroaromatic ring having one heteroatom selected from the group consisting of O, N, S and Se,
C has the general structure:
each Q comprises an aromatic substituent independently selected from the group consisting of six-membered aromatic rings, five-membered heteroaromatic rings, fused ring systems of at least one six-membered aromatic ring, and fused ring systems of at least one five-membered heteroaromatic ring having one heteroatom selected from the group consisting of O, N, S and Se,
B comprises a conjugated functional group, the value of i for each B is an integer between zero and three, inclusively, and j is from one to nine, inclusive; and
R, D, A, and B may independently be attached to a member of a heteroaromatic ring alpha to a heteroatom, and when Q is an aromatic ring, B is attached to a member of said aromatic ring para to R or another B, and
D and A can independently be ortho, meta, or para to B on Q.
D comprises an electron donating group capable of releasing electrons into said conjugated aromatic system; l is an integer between zero and three, inclusively,
A comprises an electron accepting group capable of pulling electrons from said conjugated aromatic system; m is an integer between zero and three, inclusively,

R is selected from the group consisting of straight-chained or branched alkyl, alkoxy, alkylthio, alkylamino, and fluoro-alkyl group containing from one to thirty carbon atoms attached to said composite organic compound wherein R may independently be attached to C and P by an alkyl moiety or connecting group, k is the number of R groups attached to the composite organic compound wherein R may independently be attached to C and P by an alkyl moiety or a connecting group, the value of k is an integer between 0 and 15, inclusively,
S comprises a heteroaromatic substituent selected from the group consisting of five-membered heteroaromatic rings having one heteroatom selected from the group consisting of O, N, S and Se, fused ring systems containing at least one five-membered heteroaromatic ring having one heteroatom selected from the group consisting of O, S and Se, fused ring systems containing at least one five-membered heteroaromatic ring having two to four N heteroatoms, fused ring systems containing all five-membered heteroaromatic rings having one heteroatom selected from the group consisting of O, N, S and Se, pyrimidine and purine, so that S is tricyanovinylated at a ring position alpha to a heteroatom;

P is a polycyclic conjugated molecular fragments having two-dimensional flat form and self-assembling by pi-pi stacking in a column-like supramolecule, n is the number of the polycyclic conjugated molecular fragments which is equal to 0, 2, or 4.

Another aspect of the present disclosure is to provide a capacitor with a high power output. A further aspect of the present disclosure is to provide a capacitor featuring a high dielectric constant sustainable to high frequencies. A still further aspect of the present disclosure is to provide a capacitor featuring voltage dependent capacitance. In yet another aspect of the present disclosure, a method to make such a capacitor is provided.

The capacitor, in its simplest form, comprises a first electrode, a second electrode and a composite chromophore, comprising ordered resistive tails and dopant groups, between the first electrode and the second electrode. The dopant groups on the composite chromophore can be electron acceptor and/or electron donor groups separated by a conjugated bridge. The conjugated bridge comprises one or more double bonds that alternate with single bonds in an unsaturated compound. Among the many elements that may be present in the double bond, carbon, nitrogen, oxygen and sulfur are the most preferred heteroatoms. The π electrons in the conjugated bridge are delocalized across the length of the bridge. Among the many types of ordered resistive tails that may be present in the composite chromophore, alkyl chains, branched alkyl chains, fluorinated alkyl chains, branched flouroalkyl chains, poly(methyl methacrylate) chains are examples and are preferentially positioned on the terminal aromatic rings of a chromophore. When a bias is applied across the first and second electrodes, the composite chromophore becomes more or less polarized with electron density moving from the donor to acceptor or vice versa. When the bias is removed, the original charge distribution is restored. Typically, the capacitor comprises a plurality of composite chromophores as a dielectric film with lamella or micellular structures.

In one embodiment, a liquid or solid composite chromophore is placed between the first and second electrodes. A solid chromophore is, for example, pressed into a pellet and placed between the first electrode and the second electrode. The chromophore can be ground into a powder before pressing.

In another embodiment, the composite chromophore is dissolved or suspended in a solvent. Which can be used to spin coat or pulled to form a dielectric film.

In another embodiment, the tailless composite chromophore is dissolved or suspended in a polymer. This is termed a “guest-host” system where the chromophore is the guest and the polymer is the host. Polymer hosts include, but are not limited to, poly(methyl methacrylate), polyimides, polycarbonates and poly(ε-caprolactone). These systems are cross-linked or non-cross-linked.

In another embodiment, the tailless composite chromophore is attached to a polymer. This is termed a “side-chain polymer” system. This system has the advantages over guest-host systems because high composite chromophore concentrations are incorporated into the polymer without crystallization, phase separation or concentration gradients. Side chain polymers include, but are not limited to, poly[4-(2,2-dicyanovinyl)-N-bis(hydroxyethyl)aniline-alt-(4,4′-methylenebis(phenylisocyanate))]urethane, poly[4-(2,2-dicyanovinyl)-N-bis(hydroxyethyl)aniline-alt-(isophoronediisocyanate)]urethane, poly(9H-carbazole-9-ethyl acrylate), poly(9H-carbazole-9-ethyl methacrylate), poly(Disperse Orange 3 acrylamide), poly(Disperse Orange 3 methacrylamide), poly(Disperse Red 1 acrylate), poly(Disperse Red 13 acrylate), poly(Disperse Red 1 methacrylate), poly(Disperse Red 13 methacrylate), poly[(Disperse Red 19)-alt-(1,4-diphenylmethane urethane)], poly(Disperse Red 19-p-phenylene diacrylate), poly(Disperse Yellow 7 acrylate), poly(Disperse Yellow 7 methacrylate), poly[(methyl methacrylate)-co-(9-H-carbazole-9-ethyl acrylate)], poly[(methyl methacrylate)-co-(9-H-carbazole-9-ethyl methacrylate)], poly[methyl methacrylate-co-(Disperse Orange 3 acrylamide)], poly[methyl methacrylate-co-(Disperse Orange 3 methacrylamide)], poly[(methyl methacrylate)-co-(Disperse Red 1 acrylate)], poly[(methyl methacrylate)-co-(Disperse Red 1 methacrylate)], poly[(methyl methacrylate)-co-(Disperse Red 13 acrylate)], poly[(methyl methacrylate)-co-(Disperse Red 13 methacrylate)], poly[methyl methacrylate-co-(Disperse Yellow 7 acrylate)], poly[methyl methacrylate-co-(Disperse Yellow 7 methacrylate)], poly [[(S)-1-(4-nitrophenyl)-2-pyrrolidinemethyl]acrylate], poly[((S)-1-(4-nitrophenyl)-2-pyrrolidinemethyl)acrylate-co-methyl methacrylate], poly[((S)-(−)-1-(4-nitrophenyl)-2-pyrrolidinemethyl]methacrylate] and poly[((S)-(−)-1-(4-nitrophenyl)-2-pyrrolidinemethyl)methacrylate-co-methyl methacrylate]. These systems are cross-linked or non-cross-linked. In such embodiments the chromophore may be attached to the polymer directly via a single bond or through linking groups. Such linking groups may include but are not limited to the following.

TABLE 1
Examples of Suitable Linking Groups
Between Polymer and Chromophore.

| | |
|---|---|
| —O— | 29 |
| 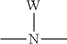 | 30 |
| 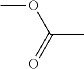 | 31 |
| 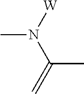 | 32 |
| 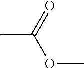 | 33 |
| 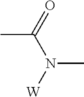 | 34 |
| 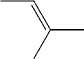 | 35 |
| 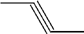 | 36 |
| 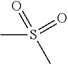 | 36 |
| 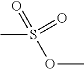 | 37 |
| 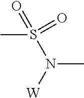 | 38 |

Where W is hydrogen (H) or an alkyl group.

In another embodiment, the tailless composite chromophore is incorporated into the polymer backbone. These systems are termed “main-chain polymer” systems. Main-chain polymers include, but are not limited to, 4-methoxy-4′-carbomethoxy-α-amino-α′-cyanostilbenes, the AB copolymer of α-cyano-m-methoxy-p-(ω-oxypropoxy)cinnamate with ω-hydroxydodecanoate, poly[(4-N-ethylene-N-ethylamino)-α-cyanocinnamate, bispheno A-4-amino-4′-nitrotolan, bisphenol A-4-nitroaniline and bisphenol A-N,N-dimethyl-4-nitro-1,2-phenylenediamine. These systems are cross-linked or non-cross-linked.

In another embodiment, the tailless composite chromophore is embedded in matrices such as oxides, halides, salts and organic glasses. An example of a matrix is inorganic glasses comprising the oxides of aluminum, boron, silicon, titanium, vanadium and zirconium.

The chromophore is aligned, partially aligned or unaligned. The composite chromophore is preferably aligned as this results in higher capacitance values in the capacitor. The preferred method of alignment is to apply a dc electric field to the composite chromophore at a temperature at which the composite chromophore can be oriented. This method is termed “poling.” Poling is generally performed near the glass transition temperature of polymeric and glassy systems. A preferred method of poling is corona poling.

A preferred capacitor further does not comprise a first insulator between the first electrode and the composite chromophore nor a second insulator between the second electrode and the composite chromophore.

Preferred electron donors include, but are not limited to, amino and phosphino groups and combinations thereof. Preferred electron acceptors include, but are not limited to, nitro, carbonyl, oxo, thioxo, sulfonyl, malononitrile, isoxazolone, cyano, dicyano, tricyano, tetracycano, nitrile, dicarbonitrile, tricarbonitrile, thioxodihydropyrimidinedione groups and combinations thereof. More conjugated bridges include, but are not limited to, 1,2-diphenylethene, 1,2-diphenyldiazene, styrene, hexa-1,3,5-trienylbenzene and 1,4-di(thiophen-2-yl)buta-1,3-diene, alkenes, dienes, trienes, polyenes, diazenes and combinations thereof.

The first and second electrodes are selected from the group consisting of conductors and semiconductors. Conductors include, but are not limited to, metals, conducting polymers, carbon nano-materials, and graphite including graphene sheets. Semiconductors include, but are not limited to, silicon, germanium, silicon carbide, gallium arsenide and selenium. The electrode may or may not be formed on a flat support. Flat supports may include, but are not limited to, glass, plastic, silicon, and metal surfaces.

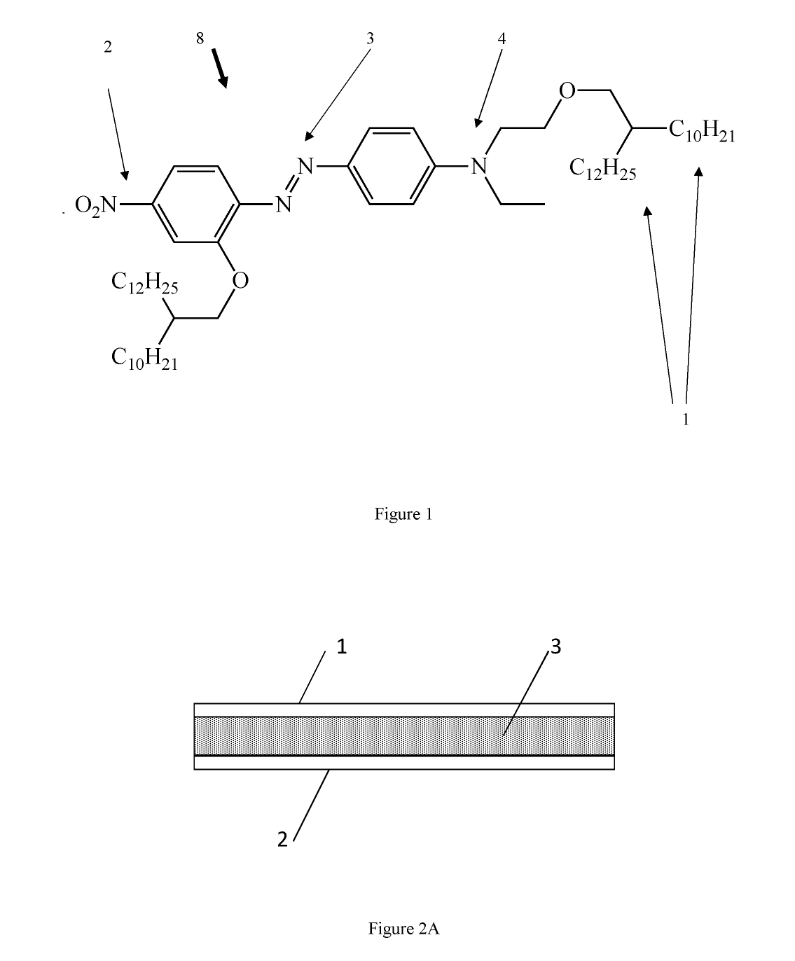

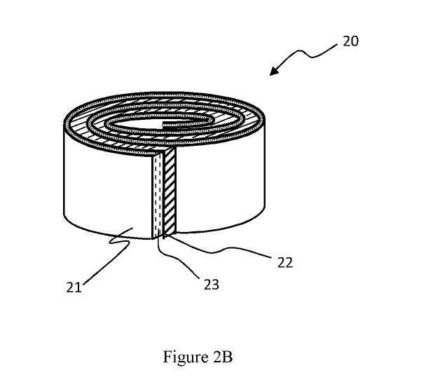

FIG. 1 illustrates the components in a chromophore 8, an electron donor 4, a conjugated bridge 3, an electron acceptor 2, and tail 1. A composite chromophore can have more than one electron donor 4, electron acceptor 2, conjugated bridge 3, and tail 1. A composite chromophore can comprise a mixture of molecules.

In one embodiment of the present disclosure, the metadielectric layer comprises the column-like supramolecules formed by the electro-polarizable compounds comprising rylene fragments of a single or a variety of lengths. Some non-limiting embodiments are shown below.

TABLE 2
Rylene fragment examples.

| | |
|---|---|
| 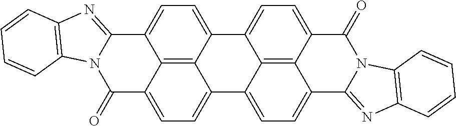 | 1 |
| 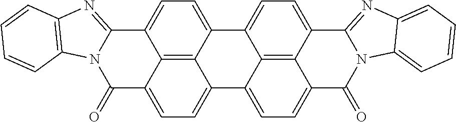 | 2 |
| 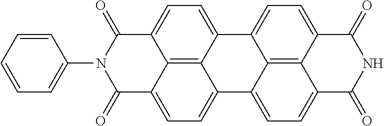 | 3 |
| 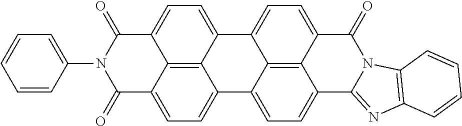 | 4 |
| 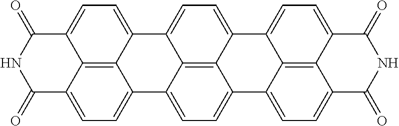 | 5 |
| 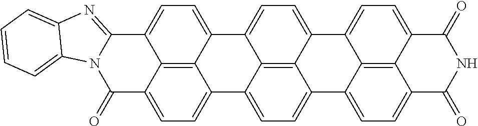 | 6 |
| 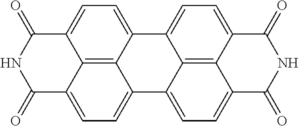 | 7 |
| 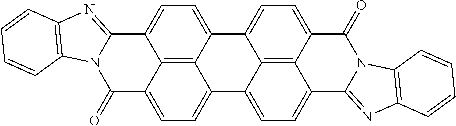 | 8 |
| 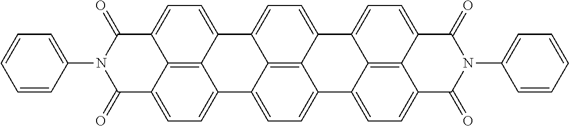 | 9 |
| 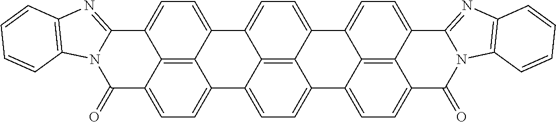 | 10 |
| 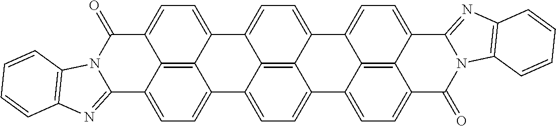 | 11 |
| 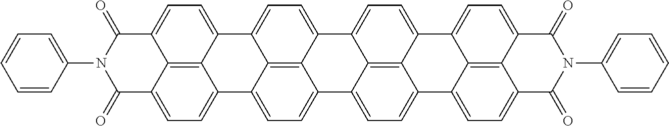 | 12 |
| 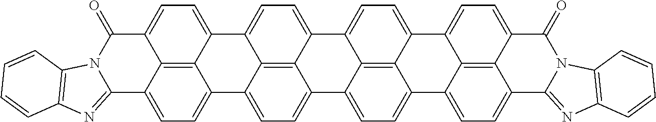 | 13 |
| 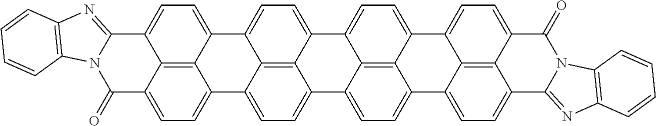 | 14 |
| 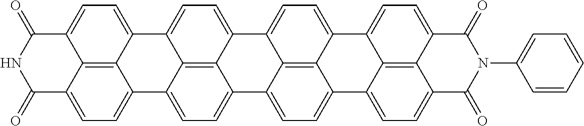 | 15 |
| 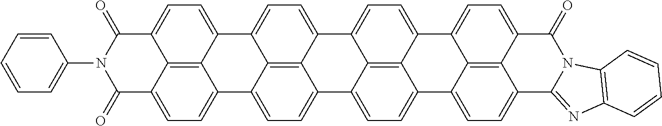 | 16 |
| 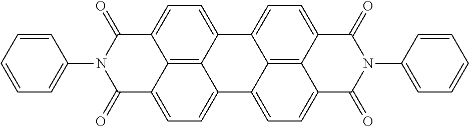 | 17 |
| 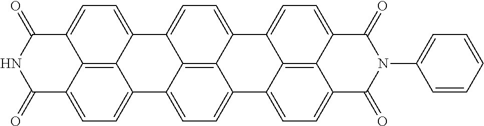 | 18 |
| 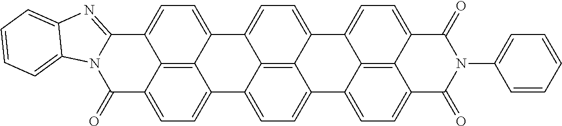 | 19 |
| 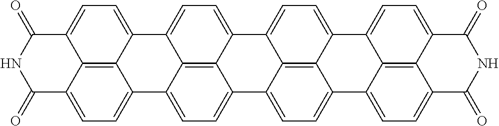 | 20 |
| 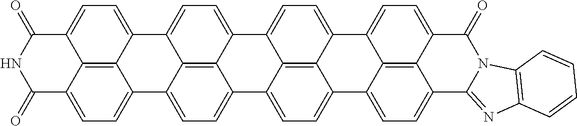 | 21 |

In one embodiment of the present disclosure, the layer's relative permittivity is greater than or equal to 1000. In another embodiment of the present disclosure, the polarization (P) of the metadielectric layer comprises first-order (ε₍₁₎) and second-order (ε₍₂₎) and third order (ε₍₃₎) permittivities according to the following formula:

P=ε₀(ε₁−1)E+ε₀ε₂E²+ε₀ε₃E³+ . . .

where P is the polarization of the material, which also can be represented by the following formula:

P=NP_{induced}

where P_{induced} is the induced polarization which can be expressed by the formula:

P_{induced}=αE_{loc}+βE_{loc}²+γE_{loc}³+ . . .

where E_{loc} is the localized field and is expressed by the formula:

E_{loc}=E+P/(3ε₀)

The real part of the relative permittivity (ε′) as can be seen from the above equations, also comprises first, second, and third order permittivities. Further, permittivity of a capacitor is a function of applied voltage and thickness of the capacitor's dielectric (d). Where voltage is the DC-voltage which is applied to the crystal metadielectric layer, and d is the layer thickness. In another embodiment of the present invention, the layer's resistivity is greater than or equal to 10¹³ ohm cm.

In one embodiment, the composite chromophore comprises more than one electron donor-conjugated bridge-electron acceptor combination in series. In another embodiment, the composite chromophore comprises more than one electron donor-conjugated bridge-electron acceptor combination in parallel. In yet another embodiment, the composite chromophore comprises electron donor-conjugated bridge-electron acceptor combinations both in parallel and in series. In still another embodiment, the composite chromophore comprises a more than one type of ordered resistive tails.

The present disclosure provides the meta-capacitor comprising two metal electrodes positioned parallel to each other and which can be rolled or flat and planar and a metadielectric layer between said electrodes. The layer comprises the electro-polarizable compounds as disclosed above.

A metadielectric layer maybe a film comprising the above described composite organic compound comprising chromophore fragments with dopants and ordered resistive tails.

The meta-capacitor comprises a first electrode 1, a second electrode 2, and a metadielectric layer 3 disposed between said first and second electrodes as shown in FIG. 1A. Electrodes 1 and 2 may be made of a metal, such as copper, zinc, or aluminum or other conductive material such as graphite or carbon nanomaterials and are generally planar in shape.

Electrodes 1 and 2 may be flat and planar and positioned parallel to each other. Alternatively, the electrodes may be planar and parallel, but not necessarily flat, they may be coiled, rolled, bent, folded, or otherwise shaped to reduce the overall form factor of the capacitor. It is also possible for the electrodes to be non-flat, non-planar, or non-parallel or some combination of two or more of these. By way of example and not by way of limitation, a spacing d between electrodes 1 and 2 may range from about 100 nm to about 100 μm. The maximum voltage V_{bd} between electrodes 1 and 2 is approximately the product of the breakdown field E_{bd} and the electrode spacing d. If E_{bd}=0.1 V/nm and the spacing d between electrodes 1 and 2 is 100 microns (100,000 nm), the maximum voltage V_{bd} would be 10,000 volts.

Electrodes 1 and 2 may have the same shape as each other, the same dimensions, and the same area A. By way of example, and not by way of limitation, the area A of each electrode 1 and 2 may range from about 0.01 m² to about 1000 m². By way of example and not by way of limitation for rolled capacitors, electrodes up to, e.g., 1000 m long and 1 m wide.

These ranges are non-limiting. Other ranges of the electrode spacing d and area A are within the scope of the aspects of the present disclosure.

If the spacing d is small compared to the characteristic linear dimensions of electrodes (e.g., length and/or width), the capacitance C of the capacitor may be approximated by the formula:

C=εε₀A/d, (V)

where εₒ is the permittivity of free space (8.85×10⁻¹² Coulombs²/(Newton·meter²)) and ε is the dielectric constant of the dielectric layer. The energy storage capacity U of the capacitor may be approximated as:

U=½εεₒAE_{bd}²d (VI)

The energy storage capacity U is determined by the dielectric constant ε, the area A, and the breakdown field E_{bd}. By appropriate engineering, a capacitor or capacitor bank may be designed to have any desired energy storage capacity U. By way of example, and not by way of limitation, given the above ranges for the dielectric constant ε, electrode area A, and breakdown field E_{bd} a capacitor in accordance with aspects of the present disclosure may have an energy storage capacity U ranging from about 500 Joules to about 2·10¹⁶ Joules.

For a dielectric constant ε ranging, e.g., from about 100 to about 1,000,000 and constant breakdown field E_{bd} between, e.g., about 0.1 and 0.5 V/nm, a capacitor of the type described herein may have a specific energy capacity per unit mass ranging from about 10 W·h/kg up to about 100,000 W·h/kg, though implementations are not so limited.

The present disclosure includes meta-capacitors that are coiled, e.g., as depicted in FIG. 1B. In this example, a meta-capacitor 20 comprises a first electrode 21, a second electrode 22, and a metadielectric material layer 23 of the type described hereinabove disposed between said first and second electrodes. Electrodes 21 and 22 may be made of a metal, such as copper, zinc, or aluminum or other conductive material such as graphite or carbon nanomaterials and are generally planar in shape. In one implementation, the electrodes and metadielectric material layer 23 are in the form of long strips of material that are sandwiched together and wound into a coil along with an insulating material, e.g., a plastic film such as polypropylene or polyester to prevent electrical shorting between electrodes 21 and 22.

In order that the invention may be more readily understood, reference is made to the following examples, which are intended to be illustrative of the invention, but are not intended to limit the scope.

### EXAMPLE 1

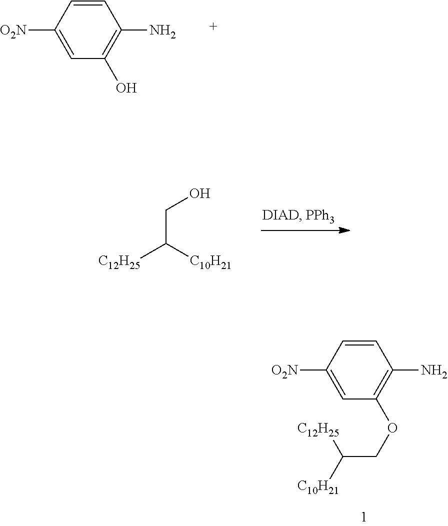

2-decyl-1-tetradecanol (1 equiv.), PPh₃ (2 equiv.), and DIAD (2.3 equiv.) were dissolved in THF and stirred in an ice bath. Then, 2-amino-5-nitrophenol was added and the reaction was allowed to warm to ambient temperature and stirred for 24 h. The reaction mixture was diluted with hexanes and filtered through diatomaceous earth. The filtrate was concentrated and purified on silica gel to give 1.

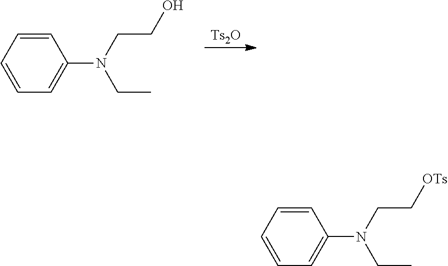 [SMILES: "Ts2O" ; CCN(CCO)c1ccccc1 ; CCN(CCOS(=O)(=O)c1ccc(C)cc1)c1ccccc1]

2-(N-ethylanilino)ethanol (1 equiv.), NaH (2 equiv.), and tosyl chloride (1.2 equiv.) were dissolved in DMF and stirred at room temperature for 18 h. The solution was processed through an aqueous workup. The organics were dried over MgSO₄ and the solvents were removed en vacuo.

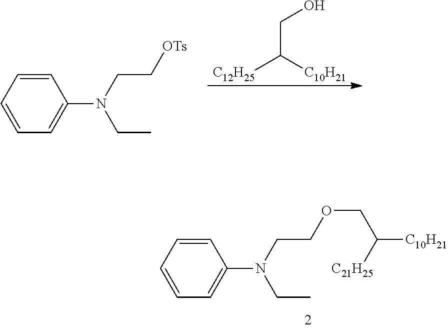

2-decyl-1-tetradecanol (1 equiv.), NaH (2 equiv.), and tosylated 2-(N-ethylanilino)ethanol (1 equiv.) were dissolved in THF and stirred at room temperature for 18 h. The solution was processed through an aqueous workup. The organics were dried over MgSO₄ and the solvents were removed en vacuo to give 2.

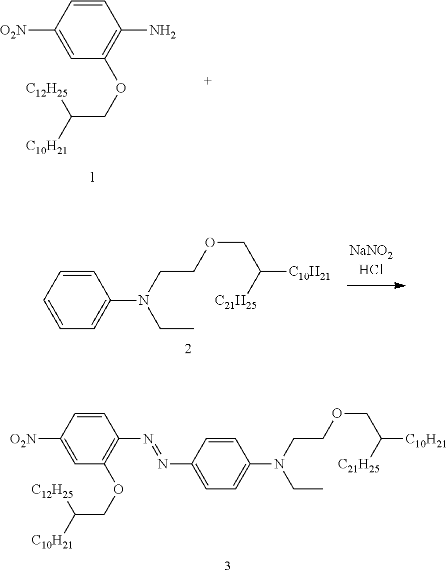

Compound 1 (20 mmol) was dissolved in a solution of 35% hydrochloric acid and the mixture was stirred in an ice bath. Subsequently, a water solution of sodium nitrite (20 mmol) was added slowly and the resulting solution was stirred in the ice bath for 30 min, a solution of 2 (24 mmol) in distilled ethanol was added dropwise and stirred for 1 h. After pH of the resulting solution was adjusted to 7.0 with potassium carbonate, the reaction was stirred for another 30 min. The red solution was diluted with CH₂Cl₂ and washed with brine and deionized water. The crude product was purified by recrystallization.

While the above is a complete description of the preferred embodiment of the present invention, it is possible to use various alternatives, modifications and equivalents. Therefore, the scope of the present invention should be determined not with reference to the above description but should, instead, be determined with reference to the appended claims, along with their full scope of equivalents. Any feature described herein, whether preferred or not, may be combined with any other feature described herein, whether preferred or not. In the claims that follow, the indefinite article “A”, or “An” refers to a quantity of one or more of the item following the article, except where expressly stated otherwise. As used herein, in a listing of elements in the alternative, the word “or” is used in the logical inclusive sense, e.g., “X or Y” covers X alone, Y alone, or both X and Y together, except where expressly stated otherwise. Two or more elements listed as alternatives may be combined together. The appended claims are not to be interpreted as including means-plus-function limitations, unless such a limitation is explicitly recited in a given claim using the phrase “means for.”

What is claimed is:

1. A composite organic compound characterized by polarizability and resistivity that has
a general structural formula: [symbol]
where C is a chromophore fragment comprises an aromatic substituent independently selected from the group consisting of six-membered aromatic rings, five-membered heteroaromatic rings, fused ring systems containing at least one six-membered aromatic ring, and fused ring systems containing at least one five-membered heteroaromatic ring having one heteroatom selected from the group consisting of O, N, S and Se,
wherein C has the general structure: [symbol]
wherein each Q comprises an aromatic substituent independently selected from the group consisting of six-membered aromatic rings, five-membered heteroaromatic rings, fused ring systems of at least one six-membered aromatic ring, and fused ring systems of at least one five-membered heteroaromatic ring having one heteroatom selected from the group consisting of O, N, S and Se,
B comprises a conjugated functional group, the value of i for each B is an integer between zero and three, inclusively, and j is from one to nine, inclusive; and
R, D, A, and B may independently be attached to a member of a heteroaromatic ring alpha to a heteroatom, and when Q is an aromatic ring, B is attached to a member of said aromatic ring para to R or another B, and
where D and A can independently be ortho, meta, or para to B on Q.
D comprises an electron donating group capable of releasing electrons into said conjugated aromatic system; l is an integer between zero and three, inclusively,
A comprises an electron accepting group capable of pulling electrons from said conjugated aromatic system; m is an integer between zero and three, inclusively,
R is selected from the group consisting of straight-chained or branched alkyl, alkoxy, alkylthio, alkylamino, and fluoro-alkyl group containing from one to thirty carbon atoms attached to said composite organic compound wherein R may independently be attached to C and P by an alkyl moiety or connecting group, k is the number of R groups attached to the composite organic compound wherein R may independently be attached to C and P by an alkyl moiety or a connecting group, the value of k is an integer between 0 and 15, inclusively,
S comprises a heteroaromatic substituent selected from the group consisting of five-membered heteroaromatic rings having one heteroatom selected from the group consisting of O, N, S and Se, fused ring systems containing at least one five-membered heteroaromatic ring having one heteroatom selected from the group consisting of O, S and Se, fused ring systems containing at least one five-membered heteroaromatic ring having two to four N heteroatoms, fused ring systems containing all five-membered heteroaromatic rings having one heteroatom selected from the group consisting of O, N, S and Se, pyrimidine and purine, so that S is tricyanovinylated at a ring position alpha to a heteroatom;
where P is a polycyclic conjugated molecular fragments having two-dimensional flat form and self-assembling by pi-pi stacking in a column-like supramolecule, n is the number of the polycyclic conjugated molecular fragments which is equal to 0, 2, or 4.

2. The composite organic compound of claim 1 wherein the chromophore further comprises more than one electron donor-conjugated bridge-electron acceptor combination in series or in parallel.

3. The composite organic compound of claim 1 wherein the chromophore (C) further comprises more than one electron donor-conjugated bridge-electron acceptor combination in series and in parallel.

4. The composite organic compound of claim 1 wherein the conjugated bridge (B) is selected from the group consisting of alkenes, dienes, trienes, polyenes, 1,2-diphenylethene, 1,2-diphenyldiazene, styrene, hexa-1,3,5-trienylbenzene, 1,4-di(thiophen-2-yl)buta-1,3-diene and combinations thereof.

5. The composite organic compound of claim 1 wherein the electron donor (D) is selected from the group consisting of —O⁻ (phenoxides, like —ONa or —OK), —NH₂, —NHR, —NR₂, —OH, —OR (ethers), —NHCOR (amides, from amine side), —OCOR (esters, from alcohol side), alkyls, —C₆H₅, vinyls, wherein R is radical selected from the list comprising alkyl (methyl, ethyl, iso-propyl, tert-butyl, neo-pentyl, cyclohexyl etc.), allyl (—CH2-CH═CH2), benzyl (—CH2C6H5) groups, phenyl (+substituted phenyl) and other aryl (aromatic) groups and combinations thereof.

6. The composite organic compound of claim 1 wherein the electron acceptor (A) is selected from the group consisting of —NO₂, —NH₃⁺ and —NR₃⁺ (quaternary nitrogen salts), counterion Cl⁻ or Br⁻, —CHO (aldehyde), —CRO (keto group), —SO₃H (sulfonic acids), —SO₃R (sulfonates), —SO₂NH₂ (sulfonamides), —COOH (carboxylic acid), —COOR (esters, from carboxylic acid side), —COCl (carboxylic acid chlorides), —CONH₂ (amides, from carboxylic acid side), —CF₃, —CCl₃, —CN, wherein R is radical selected from the list comprising alkyl (methyl, ethyl, iso-propyl, tent-butyl, neo-pentyl, cyclohexyl etc.), allyl (—CH₂—CH═CH₂), benzyl (—CH₂C₆H₅) groups, phenyl (+substituted phenyl) and other aryl (aromatic) groups and combinations thereof

7. The composite organic compound of claim 1 wherein the electron donor is an amino group, the electron acceptor is selected from the group consisting of nitro, carbonyl and cyano groups and the conjugated bridge is selected from the group consisting of alkenes, diphenyldiazene, 1,2-diphenylethene and combinations thereof.

8. The composite organic compound of claim 1 wherein the electron donor (D) and electron acceptor (A) groups are arranged on the chromophore fragment such that the fragment is non-centrosymmetric.

9. The composite organic compound of claim 1 wherein the polycyclic organic molecule fragment (P) is comprised of rylene fragments meeting the formula 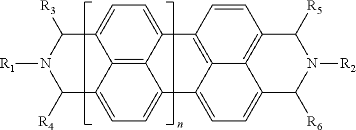
wherein n=1-10, R₃, R₄, R₅, and R₆ independently are selected from the group consisting of hydrogen atom, double bonded oxygen, and groups joining with R₁ and R₂ to form polycyclic heterocycles structures, R1 and R2 are independently selected from the group consisting of aryl, heteroaryl, or groups joining with R₃, R₄, R₅, and R₆ to form polycyclic heterocycles.

10. The composite organic compound of claim 1 wherein the polycyclic organic molecule fragment (P) is comprised of rylene fragments selected from structures 1 to 21:

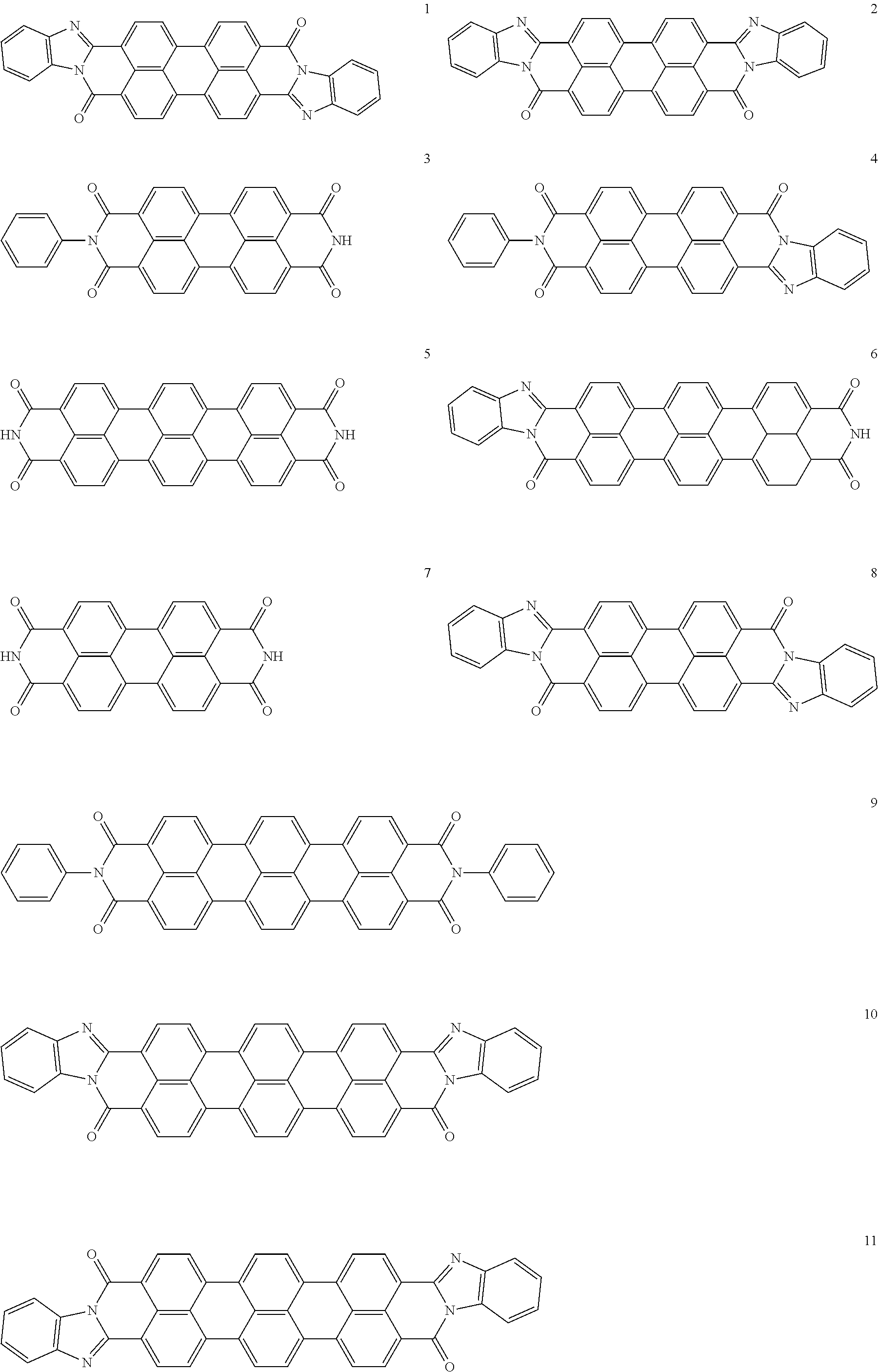

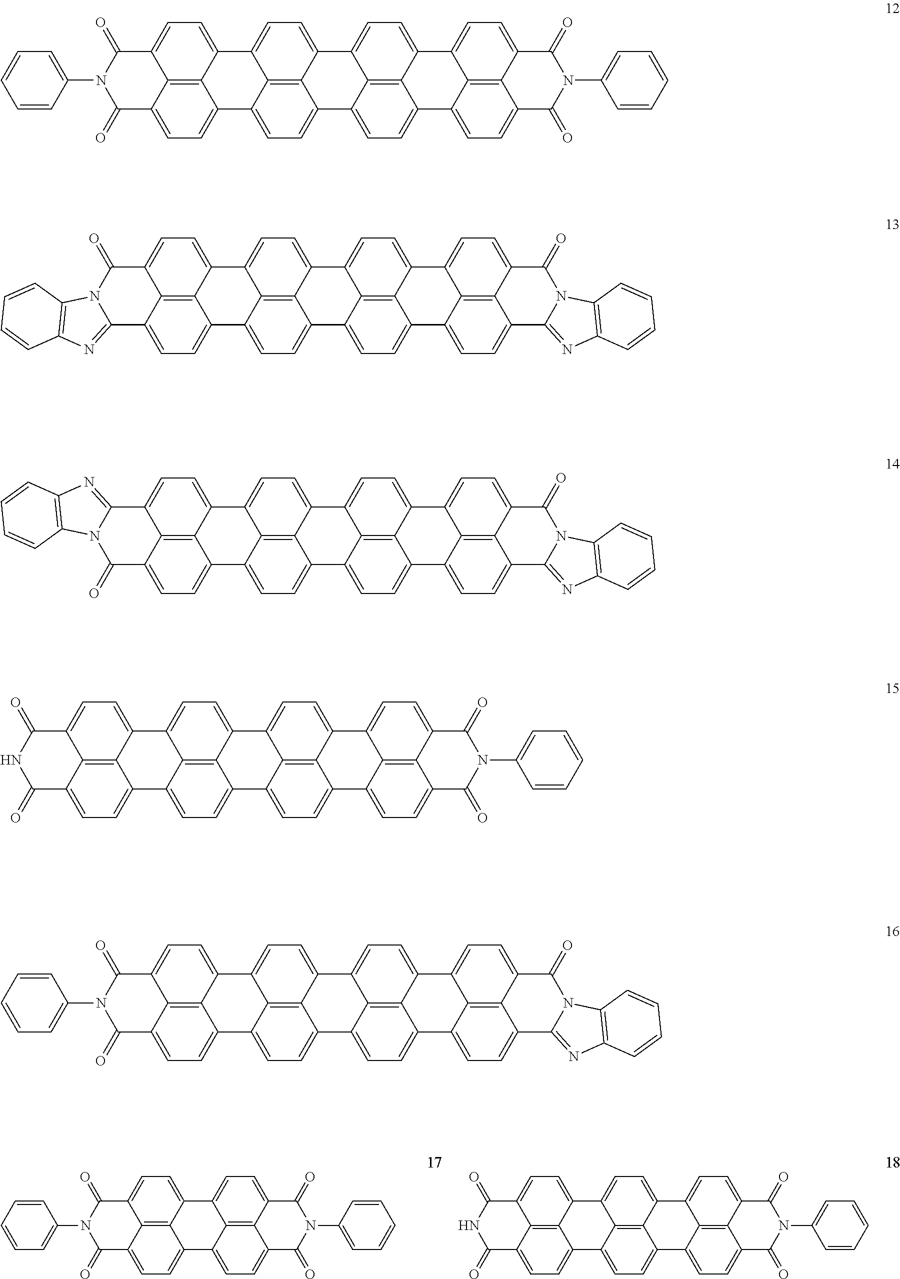

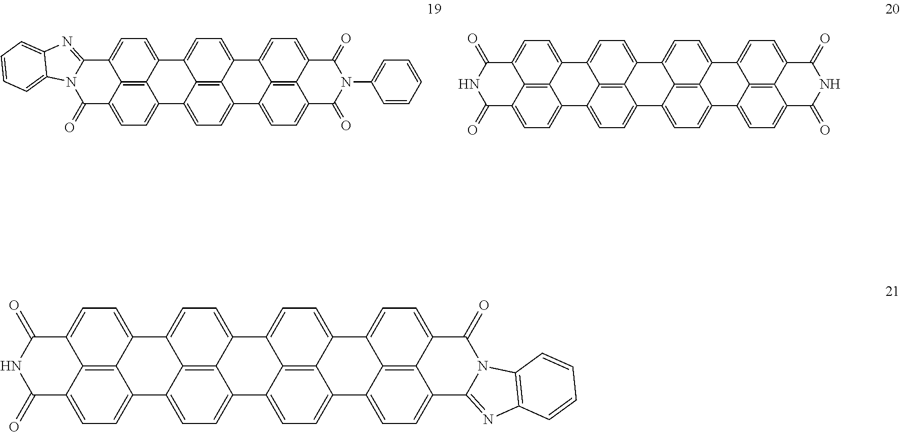 [SMILES: O=C1c2ccc3c4ccc5c6ccc7c(=O)n8c9ccccc9nc8c8ccc(c9ccc(c%10ccc(c2c3%10)C(=O)N1c1ccccc1)c4c59)c6c78 ; O=C1NC(=O)c2ccc3c4ccc5c6ccc7c8ccc9c%10c(ccc(c%11ccc(c%12ccc(c%13ccc1c2c%133)c4c%125)c6c%117)c%108)C(=O)NC9=O ; O=C1NC(=O)c2ccc3c4ccc5c6ccc7c8ccc9c%10c(ccc(c%11ccc(c%12ccc(c%13ccc1c2c%133)c4c%125)c6c%117)c8%10)c(=O)n1c2ccccc2nc91]

11. The composite organic compound of claim 1, wherein the connecting groups on R are independently selected from a single bond and the list comprising the following structures: 29-39, where W is hydrogen (H) or an alkyl group:

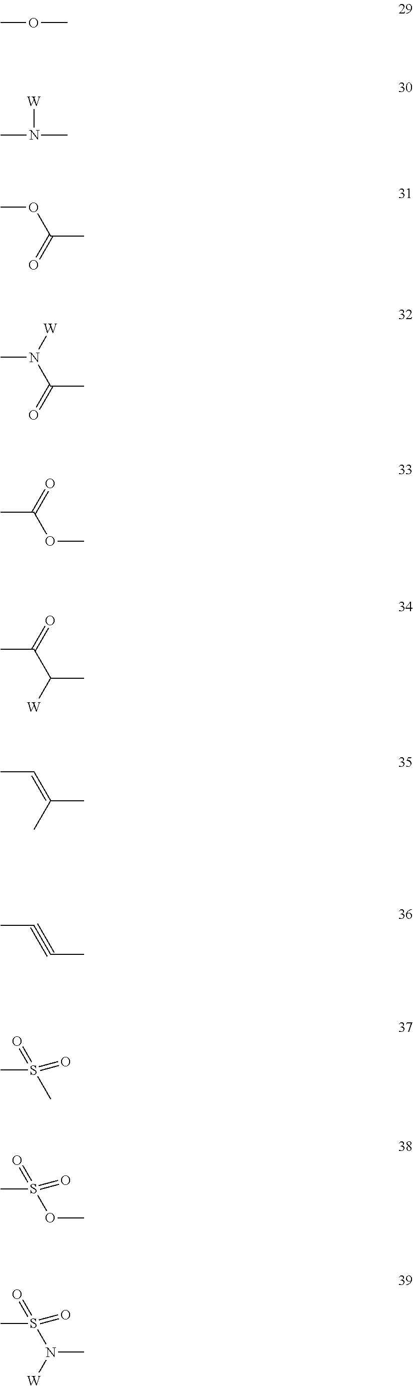

12. A metadielectric layer comprising of one or more of the type of composite organic compound as in claim 1, wherein the nonliearly polarizable fragments comprising a chromophore molecule with at least two dopant groups, the resistive dielectric envelope formed by resistive substituents providing solubility of the organic compound in a solvent and electrically insulating the column-like supramolecules from each other, and wherein the metadielectric layer's relative permittivity is greater than or equal to 1000 and resistivity is greater than or equal to 10¹⁶ Ohm cm.

13. The metadielectric layer according to claim 12, wherein the column-like supramolecules are formed by the polycyclic conjugated molecule comprising rylene fragments of same and different length.

14. A meta-capacitor comprising two metal electrodes positioned parallel to each other and which can be rolled or flat and planar and metadielectric layer between said electrodes, wherein the layer comprises the polarizable compound according to claim 1, wherein the nonlinearly polarizable fragments comprising chromophore molecule with at least one dopant group are placed into the resistive dielectric envelope formed by resistive substituents providing solubility of the organic compound in a solvent and electrically insulating the column-like supramolecules from each other.

15. A capacitor comprising:
a first electrode,
a second electrode, and
a metadielectric film as in claim 15.

16. The capacitor of claim 15 wherein the metadielectric film comprises of composite organic compound according to claim 1, and which demonstrates a non-linear effect.

17. The capacitor of claim 15 wherein the dielectric film comprises a polycyclic conjugated molecule fragment that exhibits pi-pi stacking.

18. A multilayer capacitor comprising a plurality of layers wherein each layer comprises a metadielectric film of any combination of the composite organic compounds according to claim 1 between a first electrode and a second electrode.
